(19) United States
(12) Patent Application Publication
(10) Pub. No.: US 2008/0241254 A1
(43) Pub. Date: Oct. 2, 2008

(54) PHARMACEUTICAL COMPOSITION COMPRISING ATOVAQUONE PARTICLES

(76) Inventors: Suryakant Navale, Solapur (IN); Surajit Das, Kolkata (IN); Gurvinder Singh, New Delhi (IN); Rajeev Shanker Mathur, Gurgaon (IN)

Correspondence Address: Jayadeep R. Deshmukh, Esq.;Ranbaxy Inc., Suite 2100, 600 College Road East, Princeton, NJ, 08540, US

(21) Appl. No.: 11/966,455

(22) Filed: Dec. 28, 2007

(30) Foreign Application Priority Data
Jan. 2, 2007 (IN) 10/DEL/2007

(51) Int. Cl. A61K 9/14; C07C 49/747; A61K 31/122

(52) U.S. Cl. 424/489; 568/328; 514/682

(57) ABSTRACT

The present invention relates to atovaquone particles having d₉₀ value of about 4-15 μm. It further relates to a pharmaceutical composition comprising said particles.

### FIELD OF THE INVENTION

The present invention relates to atovaquone particles having d₉₀ value of about 4-15 μm. It further relates to a pharmaceutical composition comprising said particles.

### BACKGROUND OF THE INVENTION

Many orally-administered drugs display poor bioavailability when administered in conventional dosage forms. With several drugs, absorption may be as little as 30 per cent or less of the orally administered dose. To compensate for this effect, a very large dose is often administered so that absorption of the therapeutically required quantity of the drug can occur. This technique is costly with expensive drugs, and the non-absorbed drug may also have undesirable side effects within the gastrointestinal tract. In addition, the poorly absorbed drugs often display a great deal of variability between patients in bioavailability, and this can create dosing problems. This poor bioavailability is often associated with poor solubility of drugs. There are various techniques available to overcome solubility and bioavailability problem, and one such viable technique is particle size reduction.

Atovaquone, chemically 2-[4-(4-chlorophenyl)cyclohexyl]-3-hydroxy-1,4-naphthoquinone, is a widely used antiprotozoal and is potently active (in animals and in vitro) against Pneumocystis carinii, Plasmodia, and tachyzoite and cyst forms of Toxoplasma gondii. It is a highly lipophilic compound resembling ubiquinone and has a low aqueous solubility. This is the reason for the poor bioavailability of atovaquone after oral administration. It is reported that after a single oral dose, absorption of the drug is slow and erratic; it is increased about threefold by the presence of fatty food and is dose-limited above 750 mg.

U.S. Pat. No. 4,981,874 discloses the use of atovaquone against Pneumocystis carinii infection in a mammal. EP Patent No. 0 123 238 and U.S. Pat. No. 5,053,432 disclose the use of atovaquone against Plasmodium falciparum and also against Eimeria species such as E. tenella and E. acervulina which are causative organisms of coccidiosis. Further, use of atovaquone against Toxoplasmosis and Cryptosporidiosis is disclosed in EP Patent No. 0 445 141 and 0 496 729 respectively.

Currently, atovaquone suspension marketed under trade name MEPRON is a formulation of micro-fine particles of atovaquone. The atovaquone particles are reduced in size to facilitate absorption. These particles are significantly smaller than those in the previously marketed tablet formulation. Further, U.S. Pat. Nos. 6,018,080 and 6,649,659 disclose microfluidized particles of atovaquone having improved bioavailability, wherein at least 90% of atovaquone particles have a volume diameter in the range of 0.1-3 micron.

### SUMMARY OF THE INVENTION

The present invention relates to atovaquone particles having d₉₀ value of about 4-15 μm; and pharmaceutical composition thereof.

In one aspect, there is provided a process of preparation of atovaquone particles using dry milling techniques wherein the atovaquone particles have d₉₀ value of about 4-15 μm.

In another aspect, there is provided a process of preparation of atovaquone particles using wet milling techniques wherein the atovaquone particles have d₉₀ value of about 4-15 μm.

In yet another aspect, there is provided atovaquone particles having d₉₀ value of about 4-15.

In yet another aspect, there is provided a pharmaceutical composition comprising atovaquone particles wherein the atovaquone particles have d₉₀ value of about 4-15 μm.

The pharmaceutical composition may be a solid or liquid dosage form.

According to another aspect, there is provided a process for the preparation of solid pharmaceutical composition comprising the steps of:

a) blending the atovaquone particles with other pharmaceutical excipients,

b) optionally granulating the blend,

c) lubricating the blend of step a) or granules of step b), and

d) compressing into or filling into suitable size solid dosage form,

wherein the atovaquone particles have d₉₀ value of about 4-15 μm.

In another aspect, there is provided a liquid composition comprising atovaquone particles, wherein the atovaquone particles are suspended in a suitable solvent and said particles have d₉₀ value of about 4-15 μm.

In another aspect, there is provided a method for the treatment of protozoal infection, the method comprising orally administering to a subject a pharmaceutical composition comprising atovaquone particles wherein the said particles have d₉₀ value of about 4-15 μm.

### DETAILED DESCRIPTION OF THE INVENTION

“Atovaquone” as employed herein is intended to include isomers, cis and trans forms of atovaquone, mixture thereof, and pharmaceutically acceptable salts thereof. atovaquone may be used in any polymorphic form. It may be used alone or in combination with the drug proguanil.

As used herein, the term “pharmaceutically acceptable salts” refers to inorganic base salts such as alkali metal (e.g. sodium and potassium) salts and alkaline earth metal (e.g. calcium) salts; organic base salts e.g. phenylethylbenzylamine, dibenzylethylenediamine, ethanolamine and diethanolamine salts; and amino acid salts e.g. lysine and arginine.

Conventionally known particle size analysis methods can be used suitably for determining the particle size. For example, particle size measurement can be done using light-scattering methods and turbidimetric methods, sedimentation methods, such as pipette analysis technique using an Andreassen pipette, sedimentation scales, photosedimentometers and sedimentation in a centrifugal force field, pulse methods, for example using a Coulter counter, or sorting by means of gravitational or centrifugal force.

The term “d₉ₒ value” means at least 90% of atovaquone particles have volume diameter less than specified value when measured by a light scattering method, for example, Malvern Mastersizer.

Generally, atovaquone particles having d₉₀ value of about 4-15 μm are used. In particular, the particle of atovaquone may have d₉₀ value of 4-9 μm and corresponding d₅₀ value of 1-5 μm.

Particle size reduction may be carried out using various conventionally available mills such as ball mill, an attritor mill, a vibratory mill, air jet mill or media mills such as a sand mill and a bead mill. Air jet mill can be used only for dry milling process whereas all the other mills may be used for both dry as well as wet milling. The milling may be carried out using the atovaquone alone or with other pharmaceutically acceptable excipients such as surfactants, binding agents or diluents. During wet milling, water may be used as medium for particle size reduction.

Pharmaceutical compositions as used herein include solid dosage forms such as tablets, capsules, or liquid dosage forms such as a solution or a suspension.

Pharmaceutically acceptable excipients for solid dosage form may be selected from surfactant, diluents, binders, disintegrants, lubricants, glidants.

The liquid dosage form should have suitable properties such as viscosity, taste and flavor. The pharmaceutically acceptable excipients used in liquid dosage form may be selected from suspending agents, solvents, preservatives, coloring agents, flavoring agents and sweeteners.

The surfactant may be selected from anionic, cationic or non-ionic surface-active agents or surfactants. Suitable anionic surfactants include those containing carboxylate, sulfonate, and sulfate ions such as sodium lauryl sulfate (SLS), sodium laurate, dialkyl sodium sulfosuccinates, particularly bis-(2-ethylhexyl)sodium sulfosuccinate, sodium stearate, potassium stearate, and sodium oleate. Suitable cationic surfactants include those containing long chain cations, such as benzalkonium chloride, and bis-2-hydroxyethyl oleyl amine. Suitable non-ionic surfactants include polyoxyethylene sorbitan fatty acid esters, fatty alcohols such as lauryl, cetyl and stearyl alcohols; glyceryl esters such as the naturally occurring mono-, di-, and tri-glycerides; fatty acid esters of fatty alcohols; polyglycolized glycerides such as Gelucire; polyoxyethylene-polyoxypropylene block co-polymer such as Poloxamer, polyethoxylated castor oil such as cremophor; and other alcohols such as propylene glycol, polyethylene glycol, sorbitan, sucrose, and cholesterol.

The suspending agent is selected from the group consisting of polysaccharide, such as tragacanth, xanthan gum, bentonite, acacia and lower alkyl ethers of cellulose including the hydroxy and carboxy derivatives of the cellulose ethers, vinyl polymers such as povidone, a mixture of cellulose and xanthan gum, a mixture of polyethylene glycol and sodium carboxymethyl cellulose, a mixture of xanthan gum and pregelatinized starch, a mixture of microcrystalline cellulose and sodium carboxymethyl cellulose (Avicel RC 591), and dispersed silicon dioxide (Aerosil 200). The amount of suspending agent may range from 0.01-5% w/v.

The preservative may be selected from benzyl alcohol, propylparaben, methylparaben, sorbic acid, sodium benzoate and sodium bisulphate.

The coloring agent of the present invention may be selected from any colorant used in pharmaceuticals that is approved and certified by the FDA. It may include iron oxide, lake of tartrazine, lake of quinoline yellow, lake of sunset yellow and lake of erythrosine, lake of carmosine ponceau, and allura red.

The sweetener may be selected from sucrose, lactose, glucose, aspartame, saccharine, and sorbitol solution.

The flavoring agent may be selected from yellow plum lemon, tutti frutty, aroma, peppermint oil, oil of wintergreen, cherry, orange and raspberry flavors.

The suspension may be in ready for administration form or as a powder which is to be reconstituted at the time of administration.

The suspension for oral administration is usually aqueous based wherein the suspension may comprise water, or mixture of water and one or more water-miscible solvents. Suitable water miscible solvents include propylene glycol, benzyl alcohol, ethanol and other commonly used solvents known to the skilled in the art. These solvents also act as preservatives. The viscosity of suspension may range from 300 to 3000 cps.

According to another embodiment, the atovaquone particles are prepared by:
i) Dispersing atovaquone in water;
ii) Wet milling the dispersion using dyno mill to obtain desired particle size range;
iii) Processing the resultant dispersion comprising atovaquone of desired particle size into a suitable pharmaceutical composition.

According to one of the embodiment, there is provided a process for the preparation of suspension of atovaquone comprising the steps of:
i) Reducing the particle size of atovaquone by air jet milling;
ii) Dissolving/dispersing surfactant along with other pharmaceutically acceptable excipients into a suitable solvent;
iii) Dispersing of atovaquone particles in the dispersion of step ii) under stirring;
iv) Passing the resultant mixture through a homogenizer to obtain said suspension.

A combination of dry and wet milling may also be used to achieve the desired pharmaceutical composition.

According to one of the embodiments the process for the preparation of suspension of atovaquone comprises the steps of:
i) Reducing the particle size of atovaquone by air jet milling;
ii) Dissolving/dispersing surfactant along with other pharmaceutically acceptable excipients into a suitable solvent;
iii) Dispersing of atovaquone particles in dispersion of step ii) under stirring;
iv) Passing the resultant mixture through a dyno mill to obtain said suspension.

The following examples are provided to enable one of ordinary skill in the art to prepare dosage forms of the invention and should not be construed as limiting the scope of invention.

### EXAMPLES

### Example 1

Atovaquone was air jet milled using at inlet feeder air pressure of 6 kg/cm2 and inlet miller air pressure of 8 kg/cm2 at the rate of 100 g/hour. After one cycle the contents were again milled through air jet mill for second cycle to obtain the desired particle size. The particle size of this air jet milled atovaquone particles was determined and d₉₀ was found to be 7.66 μm.

### Example 2

Atovaquone suspension was prepared using atovaquone particles of Example 1:

| S NO. | Ingredient | mg | |
|---|---|---|---|
| 1 | Atovaquone | 750 | |
| 2 | Benzyl alcohol | 50 | |
| 3 | Sodium saccharin | 30 | |
| 4 | Povidone | 25 | |
| 5 | Xanthan gum | 5 | |
| 6 | Poloxamer 188 | 25 | |
| 7 | Tutti fruiti flavor | 7.5 | |
| 8 | Water | q.s. to 5 | ml |

### Procedure:

1. Povidone and Poloxamer 188 were dissolved in part of water.
2. Sodium saccharin was dissolved in another part of water and added to solution of step 1.
3. Benzyl alcohol was added to the solution of step 2.
4. Atovaquone was dispersed in the solution of step 3.
5. Xanthan gum was added to the dispersion of atovaquone and homogenized,
6. Flavor was added to the dispersion of step 5 and homogenized again till uniform suspension is obtained.

The viscosity of atovaquone suspension was measured using Brookfield RVT model and was found to be 860 cps.

### Example 3

| S. No | Ingredients | mg/5 ml | |
|---|---|---|---|
| 1 | Atovaquone* | 750.0 | |
| 2 | Benzyl alcohol | 50.0 | |
| 3 | Sodium saccharin | 35.0 | |
| 4 | Xanthan gum | 37.5 | |
| 5 | Poloxamer 188 | 60.0 | |
| 6 | Polyoxyl 35 Castor Oil (Cremophor EL) | 60.0 | |
| 7 | Tutti fruiti flavor | 7.5 | |
| 8 | Purified water | q.s. to 5 | ml |

*Particle size of atovaquone when measured by Malvern was found to be d₉₀-5.34 μm and d₅₀-2.42 μm

### Procedure:

1. Xanthum gum was dissolved in a part of purified water under slow stirring.
2. A part of water was added to a vessel fitted with propeller type stirrer & a homogenizer.
3. Poloxamer was added to the water of step 2 under stirring.
4. Sodium saccharin was added to the solution of step 3.
5. Cremophor EL was added to the solution of step 4.
6. Benzyl alcohol was added to the solution of step 5.
7. Atovaquone was added to the solution step 6 and vacuum was applied.
8. The solution of step 1 was added to the dispersion of step 7
9. Flavors were added to the dispersion of step 8.
10. The dispersion of step 9 was homogenized using Silverson homogeniser under vacuum.
11. Volume of the dispersion of step 10 was made up with the remaining part of water.

The viscosity of atovaquone suspension was measured using Brookfield RVT model and was found to be 2470 cps.

The particle size of the homogenized atovaquone particles was determined and d₉₀ was found to be 5.45 μm and d₅₀ was found to be 2.44 μm.

### Example 4

Suspension of atovaquone may be prepared using micronized particles of Example 1:

| S NO. | Ingredient | Mg | |
|---|---|---|---|
| 1 | Atovaquone | 750 | |
| 2 | Benzyl alcohol | 50 | |
| 3 | Sodium saccharin | 30 | |
| 4 | Povidone | 25 | |
| 5 | Xanthan gum | 5 | |
| 6 | Polaxamer 188 | 25 | |
| 7 | Tutti fruiti flavor | 7.5 | |
| 8 | Water | q.s. to 5 | ml |

### Procedure:

Procedure described in Example 2 may be used and the suspension of step 6 passed through a Dyno Mill®, containing zirconium beads, as continuous process for two hours to further reduce the particle size.

### Example 5

Atovaquone particles may be prepared using using Dyno Mill®.

| S NO. | Ingredient | Qty. (g) | |
|---|---|---|---|
| 1 | atovaquone | 120 | |
| 2 | Xanthan gum | 0.3 | |
| 3 | Poloxamer 188 | 0.3 | |
| 4 | Water | q.s. to 600 | g |

### Procedure

1. Disperse Poloxamer 188 and xanthan gum in water.
2. Disperse atovaquone in the dispersion of step 1 and stir.
3. Pass atovaquone dispersion through Dyno Mill®, containing Zirconium beads, as a continuous process for 1 h.
4. Withdraw samples after regular intervals till desired particle size is obtained.

### Example 6

Suspension of atovaquone may be prepared using milled atovaquone from Example 4:

| S NO. | Ingredient | Qty (g) | |
|---|---|---|---|
| 1 | Slurry of atovaquone | 375 | |
| 2 | Benzyl alcohol | 5.0 | |
| 3 | Sodium saccharin | 0.5 | |
| 4 | Povidone | 25 | |
| 5 | Xanthan gum | 0.2 | |
| 6 | Poloxamer 188 | 0.7 | |
| 7 | Tutti fruiti flavor | 0.5 | |
| 8 | Water | q.s. to 500 | ml |

### Procedure

1. Disperse Poloxamer and xanthan gum in part of purified water.
2. Add benzyl alcohol and sodium saccharin in the dispersion of step 1.
3. Add atovaquone slurry of Example 4 into the dispersion of step 2 under homogenization till a uniform suspension is formed.

We claim:

1. Atovaquone particles having d₉₀ value of about 4-15 μm.

2. The atovaquone particles according to claim 1 wherein said particles have d₉₀ value of about 4-9 μm.

3. The atovaquone particles according to claim 2 wherein said particles additionally have d₅₀ value of about 1-5 μm.

4. The atovaquone particles according to claims 1 to 3 wherein said particles are obtained by dry or wet milling.

5. The atovaquone particles according to claim 4 wherein the milling is carried out in ball mill, attritor mill, vibratory mill, sand mill or bead mill.

6. The atorvaquone particles according to claim 1 wherein the atorvaquone particles are presented in a pharmaceutical composition.

7. The atorvaquone particles according to claim 6 wherein the pharmaceutical composition is a solid.

8. A liquid composition of atovaquone comprising atovaquone particles suspended in a solvent, wherein said particles have d₉₀ value of about 4-15 μm.

9. The liquid composition of atovaquone according to claim 8 wherein said particles have d₉₀ value of about 4-9 μm.

10. The liquid composition of atovaquone according to claim 9 wherein said particles have d₅₀ value of about 1-5 μm.

11. The liquid composition of atovaquone according to claim 8 wherein the solvent is selected from the group consisting of water and a mixture of water and one or more water-miscible solvents.

12. The liquid composition of atovaquone according to claim 11 wherein the water miscible solvents are selected from the group consisting of propylene glycol, benzyl alcohol, and ethanol.

13. The liquid composition of atovaquone according to claim 8 wherein the liquid composition further comprises pharmaceutically acceptable excipients selected from the group consisting suspending agents, surfactants, preservatives, coloring agents, flavoring agents and sweeteners.

14. The liquid composition of atovaquone according to claim 13 wherein the suspending agent is selected from the group consisting of polysaccharides, vinyl polymers, a mixture of cellulose ether and xanthan gum, a mixture of polyethylene glycol and sodium carboxymethyl cellulose, a mixture of xanthan gum and pregelatinized starch, a mixture of microcrystalline cellulose and sodium carboxymethyl cellulose, and dispersed silicon dioxide.

15. The liquid composition of atovaquone according to claim 8 wherein the suspension is prepared by a process comprising the steps of:
a) Dissolving/dispersing a surfactant along with a suspending agent and other pharmaceutically acceptable excipients into a suitable solvent,
b) Dispersing the atovaquone particles having the particle size with d₉₀ value of 4-9 μm in the dispersion of step a) under stirring,
c) Passing the resultant mixture through a homogenizer to obtain said suspension.

16. The liquid composition of atovaquone according to claim 8 wherein the suspension is prepared by a process comprising the steps of:
a) Dissolving/dispersing a surfactant along with a suspending agent and other pharmaceutically acceptable excipients into a suitable solvent,
b) Dispersing the atovaquone in the dispersion of step a) under stirring,
c) Passing the resultant mixture through a Dyno Mill® to obtain suspension comprising atovaquone having particle size with d₉₀ value of 4-9 μm.

17. The liquid composition of atovaquone according to claim 8 for the treatment of protozoal infections.
